(19) United States
(12) Patent Application Publication
(10) Pub. No.: US 2002/0006415 A1
(43) Pub. Date: Jan. 17, 2002

(54) CHEMICALLY ACTIVE FIBER COMPOSITIONS AS DELIVERY SYSTEM FOR CHEMOTHERPEUTIC AGENTS, EPECIALLY FOR SUBSTANCES USEFUL IN DENTAL HYGIENE

(76) Inventors: ROBERT R. BURCH, NEW ORLEANS, LA (US); ROBERT R. BURCH JR., EXTON, PA (US)

Correspondence Address: BRUCE M MONROE, RATNER & PRESTIA, P O BOX 7228, WILMINGTON, DE, 19803

(21) Appl. No.: 09/245,625

(22) Filed: Feb. 5, 1999

Related U.S. Application Data
(60) Provisional application No. 60/073,755, filed on Feb. 5, 1998.

(51) Int. Cl. A61K 9/00

(52) U.S. Cl. 424/400

(57) ABSTRACT

An imbibed fiber that can be used as a delivery system for chemotherapeutic agents, especially substances useful in dental hygiene, is disclosed. The imbibed fiber comprises a fiber of an elastomeric polymer capable of imbibing the chemotherapeutic agents. A preferred elastomeric fiber is spandex. The fiber may be in the form of a dental floss, dental tape, gauze pad, or the like. A therapeutically effective amount of the agent is imbibed in the elastomeric polymer.

### CROSS-REFERENCE TO RELATED APPLICATIONS

This application claims priority from U.S. Provisional Application 60/073,755, filed Feb. 5, 1998, incorporated herein by reference.

### FIELD OF THE INVENTION

This invention relates to dental hygiene. In particular, this invention relates an imbibed fiber that can be used as a delivery system for substances useful in dental hygiene.

### BACKGROUND OF THE INVENTION

Dental floss is a very well known and broadly used article of dental hygiene. There are many benefits attributed to dental flossing, and especially daily dental flossing. Dental flossing removes residual food particles, which cannot be removed by brushing, from in between teeth, and in general maintains the gums in good health. Thus, among other benefits, it decreases the incidence of dental caries, gingivitis, halitosis, and dysgeusia (bad taste). It may also reduce the incidence of plaque formation.

Providing therapeutic quantities of substances useful in dental hygiene to the interdental spaces is a desirable use of dental floss. Conventional dental floss materials, such as nylon, cotton, perfluorinated polyolefins (Teflon®, Gore-Tex®), are “hard” and, therefore, relatively impermeable. Conventional dental floss materials are, for this purpose, limited to using coatings containing the dental hygiene substance, limiting efficacy. Ashton, U.S. Pat. No. 2,667,443, for example, discloses dental floss and dental tape containing chemicals for the purpose of increasing the resistance of teeth to dental caries. King, U.S. Pat. No. 2,772,205, discloses dental floss that has absorbed thereon a higher aliphatic acyl amide of an amino carboxylic acid. Rosenberger, U.S. Pat. No. 5,280,796, discloses a dental floss coated with a microcrystalline wax containing a prophylactic, antimicrobial, analgesic and/or antiseptic material. Bowen, U.S. Pat. No. 5,603,921, discloses a method for preparing medicated dental floss in which polyethylene glycol containing an antimicrobial agent is coated onto the dental floss.

The primary impediment to patient acceptance of flossing as part of routine oral hygiene procedures is the difficulty of passing floss through tight proximal contacts. Damage to the inter proximal papilla and/or tearing or shredding of the floss are the usual reasons cited by patients for failing to use this technique.

Therefore, a need exists for a dental floss that imbibes substances useful in dental hygiene, delivers the substances in therapeutic doses to the interdental spaces where the user needs them, but is acceptable and comfortable to the user.

### SUMMARY OF THE INVENTION

The invention is a imbibed polymer, preferably an imbibed fiber, comprising:

(a) a fiber of an elastomeric polymer capable of imbibing a chemotherapeutic agent; and

(b) a therapeutically effective amount of the chemotherapeutic agent imbibed in the fiber.

Although this invention is described with respect to use of the imbibed fiber in imbibed dental floss, the invention is not limited thereby. Imbibed polymer may be in the form of, for example, a dental tape, a molded object, or a monolith. The imbibed fiber may be in the form of, for example, yarn, fabric, or a gauze pad.

### DETAILED DESCRIPTION OF THE INVENTION

The imbibed dental floss strand preferably comprises an elastomer having:

1) A tensile strength higher than 0.5 grams per denier, preferably more than 0.65 grams per denier, and more preferably more than 0.75 grams per denier.

2) The fiber requires a stress of either about 0.03 to 0.40 grams per denier, more preferably about 0.04 to 0.25 grams per denier, and even more preferably about 0.04 to 0.20 grams per denier, in order to develop an elongation of 200%, or about 0.07 to 0.60 grams per denier, more preferably about 0.08 to 0.50 grams per denier, and even more preferably about 0.09 to 0.30 grams per denier, in order to develop an elongation of 300%.

3) A break elongation of at least 200%, and more preferably of at least 300%; that is the fiber does not break before it elongates by at least that percent.

The preferred denier value for the elastomeric fiber is about 40 to 4,000, preferably about 200 to 2,500, and more preferably about 800 to 2,400.

Elastomeric fibers other than spandex fibers may be used as long as they fulfil these three important requirements along with the requirements of denier value, texture, smoothness, and the like. In addition, the fiber should preferably be stable with age. Preferably, the fiber should not lose more than 20% of its original tensile strength in one year, and more preferably it should lose not more than 10% per year.

Elastomeric dental floss is disclosed in Burch, U.S. Pat. No. 5,433,266 (equivalent to WO 95/24167), incorporated herein by reference. The dental floss comprises a fiber having a core of a segmented polymer having soft segments and hard segments. The hard segments are selected from a group consisting of urethane, amide, imide, and mixtures thereof. Urethane hard segments are, however, highly preferable. In the fiber core, the hard and soft segments belong to the same polymer and they are linked chemically by covalent bonds. The hard segments give the fibers and filaments strength and integrity. The soft segments give the fibers and filaments their elasticity.

The core of the fiber has preferably a content of hard segment of 5-40% by weight, while the soft segments are preferably selected from the group consisting of polyester, polyether, and mixtures thereof. The structure of the core of the fiber is preferably selected from the group consisting of primary monofilament, primary multifilament, primary coalesced monofilament, and combinations thereof. The core of the fiber may be uncovered or bare core, core covered with a secondary filament, or a combination thereof. The secondary filament may be a secondary monofilament, secondary multifilament, spun filament, or a combination thereof.

The content in hard segments is preferably in the range of 5-40% by weight of the total polymer, and more preferably in the range of 15-25% by weight of the total polymer. The content of the soft segment is preferably in the range of 60-95% by weight of the total polymer, and more preferably in the range of 75-85% by weight of the total polymer. Examples of such polymers are polymers having the generic name “spandex”. A review of spandex elastomeric fibers is given by L. Rose in the Reprints, Progress in Applied Chemistry (London), Vol. 51, p. 609, 1966, incorporated herein by reference. Meredith and Fyfe in Text. Inst. Ind., Vol. 2, p. 154, 1964, and Bamford in Text. Inst. Ind., Vol. 55, p. 73, 1965, discuss the structural aspects of spandex, and give suggestions about its elastomeric properties.

The hard segments of preferably aromatic polyurea are linked occasionally to the soft segments through urethane bonds, and these bonds are analogous to the covalent sulfide bonds of vulcanized rubber. The soft segments consist of a polymeric structure with a glass transition temperature well below room temperature. The soft segments in the case of spandex, which are preferably polyethers, polyesters, or a combination thereof, have preferably low internal cohesion, and they are analogous to the hydrocarbon chains in the case of natural rubber. The soft segments can dissolve other substances, such as those useful in dental hygiene.

U.S. Pat. Nos. 2,692,873, 2,751,363, 2,751,419, 3,061,574, 3,071,557, 3,133,036, and 3,149,998, British Patents 934,519 and 1,040,055, as well as Canadian Patent 692,058, all of which are incorporated herein by reference, give examples of making spandex compositions and fibers. Examples of commercial products suitable for the practice of this invention are the Lycra® spandex fibers (DuPont, Wilmington, Del.). The structure and properties of Lycra® spandex fibers are briefly discussed by Hicks in the American Dyestuff Reporter, pp. 33-35, Jan. 7, 1963. Hytrel® elastomer (DuPont, Wilmington, Del.) may also be useful, but its low content of soft segments limits the amount of substance that may be imbibed in the fiber. Natural rubber may also be useful, but its hydrocarbon-like character limits the type of substances to that can be imbibed to hydrocarbon-like small molecules and its low tensile strength is a problem in dental applications.

Unlike conventional dental floss, which has a constant diameter and may be too small for some regions between adjacent teeth and too large for other, spandex fiber has a variable diameter that depends on stretching. The diameter decreases with stretch and percent decrease in diameter related to the degree of stretch. It returns to original diameter with release of stretch. Thus, it can accommodate and clean spacings of different dimensions between teeth. Other important advantages of spandex fibers include, but are not limited to: easy and safe to use, i.e., the variable diameter with stretch enables use in variable teeth spacing; because of its good tensile strength, it does not break easily, as compared to rubber dental floss, which is prone to break easily and injure the gums of the user; once wrapped around the index finger for use, the stretch release anchors the spandex fiber with less chance of slipping, when compared to regular floss; and considerably more comfortable to the gums. Its wet smooth surface avoids gum injury. It is hypo-allergenic, and it does not have the tackiness, stickiness and unpleasant odor of rubber. It is inexpensive. It is believed to be in general more conducive to widespread flossing as compared to flossing with either non-elastic or rubber made dental floss. In contrast to rubber, it does not become brittle with time, so it may be stored for indefinite periods of time without premature breaking due to aging, which can cause gum injury during flossing. It is readily available in a variety of deniers to accommodate various interdental spacings. It is adequately elastic to reversibly stretch when subjected to a stress, but the stress required for further stretching increases considerably with elongations in the range of 200 to 400%. This, combined with the rather high tensile strength as compared to other elastomers, gives spandex fiber the robust behavior needed for its use in imbibed dental floss.

The core of the fiber may be a primary monofilament. The primary monofilament preferably has a round cross-section. However, the cross-section may have any other shape, such as for example square, rectangular, polygonal, irregular, and the like. The diameter of the primary monofilament may also be variable.

The core of the fiber may also be in the form of a primary multifilament. Monofilaments may be woven together, twisted, and in general formed in any conventional manner that fibers are manufactured. Further, the monofilaments may be coalesced into one primary coalesced monofilament, somewhat similar to the primary multifilament, with the difference that the monofilaments have been fused together to a desired degree, at least at the contact regions of the monofilaments, to form the unitary primary coalesced monofilament.

The core may be bare or covered partially or totally with a secondary monofilament or polyfilament. It may also be covered by more than one secondary mono- or multifilament. The core may be covered by a secondary spun filament. The secondary filaments may be any conventional fibers, elastomeric or non-elastic, such as, for example, nylon, acrylic, acetate, cotton, wool, polyester, Lycra® spandex, other natural or synthetic fibers, and the like.

The fiber comprises at least a therapeutically effective amount of a chemotherapeutic agent, especially a substance useful in dental hygiene. The agent preferably has sufficient solubility in water so that it can be imbibed into the fiber and extracted from the fiber when the fiber is placed in the user's mouth. More than one agent may be included, if desired.

By “therapeutically effective amount” is meant the amount necessary to achieve a desired therapeutic effect. As is well known to those skilled in the art this will depend on the agent used and the therapeutic effect desired.

Chemotherapeutic agents, especially substances useful in dental hygiene, can be imbibed into the fiber. Chemotherapeutic agents include, but are not limited to, chemical agents used as bacteriacidals, antiseptics, anti-inflammatories, anti-fungals, and agents for the treatment of cancer. These include, but are not limited to: anti-bacterials, such as sulfonamides, penicillins, trimethoprim, and, zinc salts, such as zinc acetate and zinc chloride; anti-amoebics; anti-yeasts; anti-fungals, such as nystatin and fluconazole; antiseptics, for example, fluorides, such as sodium fluoride and stannous fluoride, chlorhexidine, triclosan, thymol, chloroxylenol, hexachlorophene, phenol derivatives, such as sodium phenolate and 4-hexylresorcinol; anti-inflammatory agents, for example, salicylates, such as aspirin, phenylacetic acids, such as ibuprofen, indoleacetic acids, such as indocin, ketorolac tromethamine (Acular®), Cox 2 inhibitors, such as Celebrex®, steroids; chemotherapeutic agents (anti-neoplastic agents), such as 5-fluorouracil (5-FU), methotrexate, and cyclophosphamide; surface active agents, such as the polyethyleneimines in which 5 to 95 mole % of the nitrogen atoms have been derivatized by reaction with C₈₋₂₀ fatty acid halides and 5 to 95 mole % of the nitrogen atoms have been quaternized with HF disclosed in Homola, U.S. Pat. No. 5,665,333, incorporated herein by reference. Other materials that may be imbibed into the fiber include, for example, flavors; nascent oxygen generating agents, such as calcium peroxide; desensitizing agents, such as sodium nitrite and the barbiturates disclosed in Breuer, U.S. Pat. No. 5,252,577, incorporated herein by reference; etc. Other chemotherapeutic agents, especially those useful in dental hygiene, will be apparent to those skilled in the art.

Fluorides, such as stannous fluoride and sodium fluoride, are effective in reducing the incidence of dental carries. Although fluorinated water has been widely advocated to reduce the incidence of dental carries, only about 65% of the United States population receive fluorinated water.

The extent of caries control is generally directly related to the number of times the fluoride is applied and the length of time the fluoride is in contact with the teeth. It is better to apply lower concentrations of fluoride to the teeth more often than to apply higher concentrations at longer intervals. Fluorinated dental floss is a convenient and effective delivery system for frequent applications of low levels of fluoride. Z. Us, C. Ören, T. Ulusu, and T. Orbey, J. Dent. Child., 62(4), 274-278 (1995), concluded that use of fluoride containing dental floss is an effective method for increasing the fluoride content of interproximal surfaces, which are difficult to access and vulnerable to dental carries.

Chlorhexidine, a cationic biguanide microbiocide with a broad spectrum of activity against many forms of bacteria and fungi, is a common antibacterial agent widely used in both clinical and domestic applications. It is effective in reducing the activity of many common strains of oral flora. Chlorhexidine rinse is currently considered to be the most effective rinse to control plaque bacteria. Chlorhexidine has been a popular agent in many studies of gingivitis reversal and prevention and is used to overcome the ill effects of chronic gingivitis. However, when chlorhexidine is applied as an oral rinse, the contact time between the chlorhexidine and the oral cavity is very brief and much of the microbiocide agent is not retained. Bis-biguanides are described, for example, in Gundersen, U.S. Pat. No. 4,022,834, and Eustis, III, U.S. Pat. No. 4,053,636, incorporated herein by reference.

Surprisingly, it was found that although polyamide (nylon) fiber weakly imbibes sodium fluoride, it strongly imbibes penicillin. Although this invention is not bound by any particular theory or explanation, it is believed that the active amine ends in the nylon take up penicillins that are active toward amine chemical functionality. Any polyamide polymer, such as nylon 6,6, nylon 6, nylon 12, nylon 6,12, etc., in the form a resin, fiber, yarn, gauze, etc., can be used as a delivery system for penicillin.

The fiber may also comprise abrasive matter included within the fiber, on the surface of the fiber, or a combination thereof. The abrasive matter may be incorporated into any part of the body of the fiber, such as for example the primary or secondary filaments or both. It may also be incorporated on the surface of any or all the primary or secondary filaments, by any well known to the art techniques. When the abrasive matter is incorporated in the body of the filaments, and especially filaments of the core, care should be taken not to overfill the filament with abrasive matter to the point that it loses its strength and elasticity.

Any conventional polishing matter, used for example for polishing teeth, or mixture thereof, may be employed as an abrasive matter for the purposes of the present invention. This includes, but is not limited to appropriate oxides, such as aluminum oxide and silicon dioxide, silicates, carbonates, such as calcium carbonate, phosphates, and the like. Preferable materials are pumice, talc, calcium silicate, calcium carbonate, and zirconium oxide. The average particle size is preferably lower than 50 micrometers, and more preferably in the range of 1 to 15 micrometers.

The fiber may also include a wetting agents, such as detergents and surfactants; flavor oils, such as peppermint oil; pigments and/or coloring materials, such as titanium dioxide; antioxidants; etc.

The imbibed dental floss of this invention comprises much higher levels of chemotherapeutic agent than could be previously obtained. This high uptake is a consequence of the filament and the substance used. These high levels are by imbibing the substance or substances into the fiber instead of coating them on the surface of the fiber. For example, the dental floss comprises at least about 500 ppm fluoride, preferably at least about 1,000 ppm fluoride, more preferably at least about 1,500 ppm fluoride, and most preferably at least about 2,000 ppm fluoride. The imbibed fluoride is water soluble, so it is extracted from the imbibed dental floss during use. As discussed below in Comparative Example 1, treatment of conventional dental floss with fluoride containing solutions produces dental floss that comprises less than 500 ppm fluoride.

The chemotherapeutic agent or agents may be conveniently imbibed into the fiber to form the imbibed dental floss by dissolving the substance or substances in a hot solvent, adding the fiber to the hot solution, and allowing the solution to cool. Strongly acidic solutions, such as 0.1 M phosphoric acid (pH about 1), and long treatment times, e.g., twenty four hours or longer, are not required to achieve high levels of uptake of substances such as fluoride. Solutions or dispersions with a pH greater than about 2, preferably greater than about 3, and treatment times less than twenty four hours, preferably about 12 hours of less, may be used.

Other solvents, such as lower alcohols and mixtures of lower alcohols and water may be used. Hot water will typically be the solvent of choice, because water is non-toxic and non-flammable. The substance will typically be soluble or dispersible in water. Alternatively, if the substance is not stable to heat, the substance may be imbibed into the fiber by allowing the fiber to stand in a dilute aqueous solution of the substance, allowing diffusion to occur.

### INDUSTRIAL APPLICABILITY

The imbibed polymer may be in the form of a shaped article, such as a dental tape; a monolith; or a fiber. Imbibed fiber may be in the form of a dental floss, a gauze pad, or any other from useful for the delivery of chemotherapeutic agent, especially substances useful in dental hygiene.

When imbibed dental floss is used as a delivery system for substances useful in dental hygiene, the user of the dental floss stretches the fiber to the desired degree and forces it through two adjacent teeth. In sequence, the user reciprocates the floss of this invention back and forth. During this process the floss becomes wet, and the substance or substances useful in dental hygiene imbibed in the fiber are extracted from the floss. Stretching of the fiber promotes egress of the substance or substances, enhancing their delivery. This process is continued for a time sufficient to extract most or all of the substance or substances from the imbibed dental floss. This time will depend on the nature and amount of substance or substances present in the imbibed dental floss.

The imbibed dental floss may be in the form of a continuous strand, or a strand cut into adequately long pieces for flossing the teeth of a person. The imbibed dental floss or strand may be assembled in a dispensing box so that the fiber is at least partially enclosed in the box for purposes of cleanliness and convenience. The dispensing box typically has a means for the user to cut a continuous strand to the desired length. Such boxes are well known in the art.

The imbibed dental floss may also be assembled in combination with a dental instrument, so that the fiber is supported by the dental instrument in a manner to facilitate flossing of a person's teeth. The dental instrument is preferably disposable and made of plastic material. Other types of dental instruments are exemplified in U.S. Pat. Nos. 3,236,247, 5,010,906, 5,267,579, and 5,280,797, which are incorporated herein by reference.

Imbibed fiber in the form of a gauze pad or a specially shaped article containing imbibed polymer may be placed in the mouth and applied to the area in need of therapy, such as for example, the teeth, gums, mucosal membranes, or the mouth in general.

The advantageous properties of this invention can be observed by reference to the following examples which illustrate, but do not limit, the invention.

### EXAMPLES

### Example 1

This example illustrates preparation of a fluoride imbibed dental floss of the invention.

Lycra® spandex (12.3 g, 540 denier) was treated as follows. A solution of 14.0 g of sodium fluoride in 200 mL of deionized water was heated to 95° C. This solution was poured onto the spandex. The spandex was soaked in the solution for 30 min as the solution cooled to room temperature. The fluoride solution was decanted from the spandex, and the spandex was washed with three 200 mL portions of deionized water and dried for 4 hr under vacuum. This sample of sodium fluoride imbibed dental floss analyzed for 0.23% (2300 ppm) fluoride, indicating a very high uptake of sodium fluoride.

### Comparative Example 1

This example illustrates preparation of sodium fluoride treated conventional dental floss.

Conventional dental floss (12.0 g, Treasury™ Brand Un-Waxed, Un-Flavored) was treated as follows. A solution of 14.0 g of sodium fluoride in 200 mL of deionized water was heated to 95° C. This solution was poured onto the floss. The floss was soaked in the solution for 30 min as the solution cooled to room temperature. The fluoride solution was decanted from the floss, and the floss was washed with three 200 mL portions of deionized water and dried for 4 hr under vacuum. This sample of sodium fluoride imbibed dental floss analyzed for 385 ppm of fluorine, indicating a very poor uptake of sodium fluoride.

J. Jøgensen, M. Shariati, C. P. Shields, D. P. Durr, and H. M. Proskin, Pediatric Dentistry, 11(1), 17-20 (1989) measured the uptake of fluoride by soft filament dental floss (Super-Floss®—Oral-B Laboratories) from a fluoride containing mouthwash and a fluoride containing toothpaste. The reported results, summarized below, are similar to those reported above.

$$\begin{matrix}Impregnating Agent & ppm fluoride \\ NaF mouthwash ( 225 ppm F ) & 201 \pm 19 ppm\end{matrix}$$

$$\underline{\begin{matrix}F dentrifice slurry ( 275 ppm F ) & 248 \pm 17 ppm\end{matrix}}$$

### Example 2

This example illustrates the dental floss of the invention as a delivery system for fluoride.

Sodium fluoride imbibed spandex dental floss (2.71 g) formed in Example 1 was soaked in 12.0 g of deionized water at room temperature for 1 min. The water analyzed for 220 ppm of fluorine as sodium fluoride.

Sodium fluoride imbibed spandex dental floss (2.55 g) formed in Example 1 was soaked in 12.0 g of deionized water at room temperature for 2 min. The water analyzed for 330 ppm of fluorine as sodium fluoride.

Sodium fluoride imbibed spandex dental floss (2.58 g) formed in Example 1 was soaked in 12.0 g of deionized water at room temperature for 60 min. The water analyzed for 309 ppm of fluorine as sodium fluoride.

For reference, the deionized water analyzed for 3 ppm of fluorine as sodium fluoride, and the fluoride-treated local municipal water supply analyzed for 4 ppm of fluorine as sodium fluoride.

These results show that spandex dental floss imbibed with sodium fluoride is an effective delivery system for sodium fluoride.

### Comparative Example 2

This example illustrates extraction of sodium fluoride from conventional dental floss treated with sodium fluoride.

Sodium fluoride treated dental floss (2.52 g) formed in Comparative Example 1 was soaked in 13.2 g of deionized water at room temperature for 1 min. The water analyzed for 58 ppm of fluorine as sodium fluoride.

Sodium fluoride treated dental floss (2.52 g) formed in Comparative Example 1 was soaked in 13.2 g of deionized water at room temperature for 2 min. The water analyzed for 37 ppm of fluorine as sodium fluoride.

These results show that conventional dental floss is ineffective for delivering therapeutic doses of sodium fluoride.

### Example 3

This example illustrates preparation of penicillin containing dental floss.

Penicillin G potassium solution was prepared with a concentration of 15,000 units/cc by diluting 5 million units of penicillin as a powder in 20 cc of sterile water and diluting 15 cc of this solution to 250 cc. This was done under clean, but not sterile, conditions.

Filaments tested were: spandex (Lycra® spandex denier 2240), spandex with the coating of silicone oil coating removed, non-waxed nylon dental floss, and Hytrel® elastomer. The silicone oil coating was removed by placing the Lycra® spandex in water at 100° C. for 5 minutes, as described by the manufacturer.

Three inch segments of each of the filaments were treated under each of the following conditions:

1. Soaked in the penicillin solution at room temperature for 30 minutes.

2. Soaked in the penicillin solution at 100° C. for 30 minutes.

3. Soaked in the penicillin solution at room temperature for 6 hours.

4. Control—no treatment at all.

After treatment, each sample was washed in distilled water by swishing the fiber back and forth three times before allowing the filaments to dry in room air. The samples were stored for eight days prior to testing.

Plain Meuller Hinton agar plates were plated with penicillin sensitive Staph Aureus strain ATCC 25923 bacteria. A strand of each of the fibers was placed in each plate and the plates incubated for 24 hr and then read.

The degree of antibiotic activity was determined by the size of the area around the fiber than showed no growth of bacteria. Results were graded from negative (−), meaning no impairment of bacterial growth, to 3+, meaning marked impairment of bacterial growth. The results are shown in the following table were determined by a visual comparison of two observers.

| Sample | 30 min@25° C. | 30 min@100° C. | 6 hr@25° C. | Control |
|---|---|---|---|---|
| spandex | 2+ | 3+ | 2+ | — |
| spandex | 1+ | 2+ | 2+ | — |
| (less oil finish) | | | | |
| nylon no-wax | 3+ | 3+ | 2+ | — |
| floss | | | | |
| Hytrel ® | 1+ | 1+ | — | — |
| filament | | | | |

The untreated control had no observable effect on bacterial growth. All the imbibed dental flosses markedly arrested bacterial growth in a pattern radiating from the filament, indicating outward diffusion of penicillin. The size of the area of arrested bacterial growth was, within observable limits, a function of imbibing time.

Having described the invention, we now claim the following and their equivalents.

What is claimed is:

1. An imbibed fiber comprising:
(a) a fiber of an elastomeric polymer capable of imbibing a chemotherapeutic agent; and
(b) a therapeutically effective amount of the chemotherapeutic agent imbibed in the fiber.

2. The imbibed fiber of claim 1 in which the fiber has a core of a segmented polymer; the segmented polymer has soft segments and hard segments; the hard segments are selected from the group consisting of urethane, amide, imide, and mixtures thereof; the soft segments are selected from the group consisting of polyester, polyether, and mixtures thereof; and the hard segments are linked to the soft segments by covalent bonds.

3. The imbibed fiber of claim 2 in which the chemotherapeutic agent is a substance useful in dental hygiene.

4. The imbibed fiber of claim 1 in which the fiber has:
a denier value in the range of 40 to 4,000;
a tensile strength higher than 0.5 grams per denier; and
a break elongation of at least 400%;
the fiber requiring a stress to elongate selected from the group consisting of 0.03 to 0.4 grams per denier to develop an elongation of 200% and 0.07 to 0.6 grams per denier to develop an elongation of 300%.

5. The imbibed fiber of claim 5 in which the fiber has a core of a segmented polymer; the segmented polymer has soft segments and hard segments; the hard segments are selected from the group consisting of urethane, amide, imide, and mixtures thereof; and the hard segments are linked to the soft segments by covalent bonds.

6. The imbibed fiber of claim 5 in which the chemotherapeutic agent is a substance useful in dental hygiene.

7. The imbibed fiber of claim 5 in which the chemotherapeutic agent is selected from the group consisting of sodium fluoride and stannous fluoride.

8. The imbibed fiber of claim 5 in which the chemotherapeutic agent is a penicillin.

9. The imbibed fiber of claim 5 in which the chemotherapeutic agent is chlorhexidine.

10. An assembly comprising a box and an imbibed dental floss at least partially enclosed in said box, the imbibed dental floss comprising:
(a) a fiber of an elastomeric polymer capable of imbibing a chemotherapeutic agent; and
(b) a therapeutically effective amount of the chemotherapeutic agent imbibed in the fiber.

11. The assembly of claim 10 in which the fiber has a core of a segmented polymer; the segmented polymer has soft segments and hard segments; the hard segments are selected from the group consisting of urethane, amide, imide, and mixtures thereof; the soft segments are selected from the group consisting of polyester, polyether, and mixtures thereof; and the hard segments are linked to the soft segments by covalent bonds.

12. The assembly of claim 11 in which the fiber has:
a denier value in the range of 40 to 4,000;
a tensile strength higher than 0.5 grams per denier; and
a break elongation of at least 400%;
the fiber requiring a stress to elongate selected from the group consisting of 0.03 to 0.4 grams per denier to develop an elongation of 200% and 0.07 to 0.6 grams per denier to develop an elongation of 300%.

13. The assembly of claim 12 in which the fiber comprises at least about 2,000 ppm of water soluble fluoride.

14. A fluoride-containing fiber prepared by adding a fiber to an aqueous solution or dispersion of a fluoride salt for a time sufficient for the fiber to imbibe fluoride;
in which:
the pH of the aqueous solution or dispersion is greater than about 1; and
the fluoride-containing fiber comprises at least about 1,000 ppm of water soluble fluoride.

15. The fluoride-containing fiber of claim 14 in which the fiber comprises at least about 2,000 ppm of water soluble fluoride.

16. The fluoride-containing fiber of claim 14 in which the time sufficient for the fiber to imbibe fluoride is less than twenty four hours.

17. The fluoride-containing fiber of claim 15 in which the fiber has:
a denier value in the range of 40 to 4,000;
a tensile strength higher than 0.5 grams per denier; and
a break elongation of at least 400%;
the fiber requiring a stress to elongate selected from the group consisting of 0.03 to 0.4 grams per denier to develop an elongation of 200% and 0.07 to 0.6 grams per denier to develop an elongation of 300%.

18. The fluoride-containing fiber of claim 17 in which the fiber comprises at least about 2,000 ppm of water soluble fluoride.

19. A method for preparing a fluoride-containing fiber, the method comprising adding a fiber to an aqueous solution or dispersion of a fluoride salt for a time sufficient for the fiber to imbibe fluoride;
in which:
the pH of the aqueous solution or dispersion is greater than about 1; and
the fluoride-containing fiber comprises at least about 1,000 ppm of water soluble fluoride.

20. A method for preparing an imbibed fiber of an elastomeric polymer capable of imbibing a chemotherapeutic agent comprising a therapeutically effective amount of the chemotherapeutic agent, the method comprising adding a fiber to an aqueous solution or dispersion of a chemotherapeutic agent for a time sufficient for the fiber to imbibe the therapeutically effective amount of the chemotherapeutic agent;
in which:
the fiber has a denier value in the range of 40 to 4,000, a tensile strength higher than 0.5 grams per denier, and a break elongation of at least 400%;
the fiber requires a stress to elongate selected from the group consisting of 0.03 to 0.4 grams per denier to develop an elongation of 200% and 0.07 to 0.6 grams per denier to develop an elongation of 300%.

21. The method of claim 20 in which the aqueous solvent is water.

22. The method of claim 20 in which the fiber has a core of a segmented polymer; the segmented polymer has soft segments and hard segments; the hard segments are selected from the group consisting of urethane, amide, imide, and mixtures thereof; the soft segments are selected from the group consisting of polyester, polyether, and mixtures thereof; and the hard segments are linked to the soft segments by covalent bonds.

23. An imbibed polymer comprising:
(a) a polymer capable of imbibing penicillin; and
(b) a therapeutically effective amount penicillin imbibed in the polymer.

24. The imbibed polymer of claim 23 in which the polymer is nylon.
